(19) United States
(12) Patent Application Publication
(10) Pub. No.: US 2007/0059354 A1
(43) Pub. Date: Mar. 15, 2007

(54) SUSTAINED RELEASE DOSAGE FORMS OF OXCARBAZEPINE

(76) Inventors: Sankar Ramakrishnan, Tamil Nadu (IN); Narayanan Badri Viswanathan, Tamil Nadu (IN); Rajeev Singh Raghuvanshi, Gurgaon (IN); Ashok Rampal, Punjab (IN)

Correspondence Address: RANBAXY INC., 600 COLLEGE ROAD EAST, SUITE 2100, PRINCETON, NJ, 08540, US

(21) Appl. No.: 11/460,712

(22) Filed: Jul. 28, 2006

(30) Foreign Application Priority Data
Jul. 29, 2005 (IN) 2014/DEL/2005

(51) Int. Cl. A61K 31/55; A61K 9/48; A61K 9/22

(52) U.S. Cl. 424/451; 424/468; 514/217

(57) ABSTRACT

This invention relates to a once a day sustained release dosage form suitable for oral administration of oxcarbazepine. The once a day sustained release dosage form of oxcarbazepine includes oxcarbazepine and hydroxypropyl methylcellulose (HPMC) having a viscosity of 11,000 to 25,000 cps.

### TECHNICAL FIELD OF THE INVENTION

This invention relates to a once a day sustained release dosage form suitable for oral administration of oxcarbazepine.

### BACKGROUND OF THE INVENTION

Oxcarbazepine is an anticonvulsant and mood-stabilizing drug, used primarily in the treatment of epilepsy and bipolar disorder. It is indicated for the treatment of partial-onset seizures with or without secondarily generalized tonic-clonic seizures, in both adults and children aged over 6 years, as monotherapy or adjunctive therapy. Oxcarbazepine has been commercialized for over ten years as an immediate release formulation under the registered trademark Trileptal™.

Oxcarbazepine, a carbamazepine derivative, is practically insoluble in water, which results in poor bioavailability. One of the earlier attempts to enhance the dissolution rate and bioavailability of oxcarbazepine relied on particle size reduction of the oxcarbazepine to an order of 2 to 12 μm. A PCT application, WO 98/35681, is an illustration of oxcarbazepine composition for oral administration employing micronized drug particles of 2 to 12 μm ranges.

Our earlier PCT application WO 02/94774 describes a dosage form composition for oral administration comprising oxcarbazepine and a wetting agent. The use of wetting agents with oxcarbazepine having a median particle size of about 20 μm to about 50 μm with a maximum residue of about 10% on a 45 μm to up to 100 μm sieve gave the desired solubility and bioavailability.

The currently available oral dosage forms, such as film-coated tablets and liquid dosage forms e.g. 6% oral suspension, of oxcarbazepine are suitable for ensuring appropriate concentrations of oxcarbazepine in the blood by repeated administration over a prolonged period of treatment. Despite the merits of the known oral dosage forms, there remains a need to improve upon existing oral dosage forms of oxcarbazepine. One of the problems that may occur is the fluctuation of blood levels of oxcarbazepine on repeated administration, which may be associated with the side effects. The availability of an extended release formulation of oxcarbazepine tablets would add value to this already useful antiepileptic drug by providing the convenience of once daily dosing with expected improvement in patient compliance, an improved tolerability profile resulting from lower peak plasma levels, and a better seizure control from fewer fluctuations in the plasma levels.

PCT application WO 04/026314 discloses an oral dosage form comprising oxcarbazepine adapted to be administered once a day. U.S. Pat. No. 6,296,873 relates to a zero-order sustained release delivery system purported to be suitable for administration of carbamazepine and its derivatives. The system is so formulated that the drug is characterized as being released from the matrix at predictable, controllable, continuous, zero-order release kinetics.

### SUMMARY OF THE INVENTION

In one general aspect there is provided a once a day sustained release dosage form comprising oxcarbazepine and hydroxypropyl methylcellulose (HPMC), wherein the HPMC has a viscosity of 11,000 to 25,000 cps.

Embodiments of the dosage form may include one or more of the following features. For example, the concentration of hydroxypropyl methylcellulose may comprise 10 to 50% of the total composition. The median particle size of oxcarbazepine may be less than 30 μm. The dosage form may further comprise one or more pharmaceutically acceptable inert excipients selected from diluents, binders, disintegrants, surfactants, lubricants/glidants, and coloring agents.

The dosage form may be a tablet, capsule or pill. The dosage form may be further coated with a functional or nonfunctional coating.

The sustained release dosage form may include (a) about 40-70% by weight of oxcarbazepine, (b) about 20-40% by weight of HPMC, (c) about 1-20% by weight of diluent, and (d) about 1-20% by weight of binder. The HPMC has a viscosity of 11,000 to 25,000 cps.

In another general aspect there is provided a process for the preparation of once a day sustained release dosage form of oxcarbazepine. The process includes the steps of (a) blending oxcarbazepine and HPMC with one or more pharmaceutically acceptable inert excipients, (b) optionally granulating the blend, (c) optionally blending the granules with one or more pharmaceutically acceptable inert excipients, (d) lubricating the blend of step a) or c) or granules of step b), and (e) compressing into or filling into suitable size solid dosage form. The HPMC has a viscosity of 11,000 to 25,000 cps.

Embodiments of the process may include one or more of the following features. For example, the granulation may be wet granulation or dry granulation. The wet granulation may be with a water or suitable organic solvent or a mixture thereof.

In another general aspect there is provided a method of treating partial seizures with epilepsy comprising administering to the mammal an effective amount of a sustained release dosage form of oxcarbazepine comprising oxcarbazepine and hydroxypropyl methylcellulose (HPMC), wherein the HPMC has a viscosity of 11,000 to 25,000 cps.

The details of one or more embodiments of the inventions are set forth in the description below. Other features, objects and advantages of the inventions will be apparent from the description and claims.

### DETAILED DESCRIPTION OF THE INVENTION

The present invention provides an alternative once a day sustained release dosage form of oxcarbazepine suitable for oral administration. In the present invention, we have surprisingly discovered that a particular viscosity range of hydroxypropyl methylcellulose (HPMC) gives desired sustained release properties.

Hence, according to one of the aspects of the present invention, there is provided a once a day sustained release dosage form of oxcarbazepine comprising oxcarbazepine and HPMC wherein the HPMC has a viscosity of 11,000 to 25,000 cps.

According to another aspect of the present invention, there is provided a process for the preparation of a once a day sustained release dosage form of oxcarbazepine comprising oxcarbazepine and HPMC comprising the steps of:
a) blending oxcarbazepine and HPMC having viscosity of about 11,000 to 25,000 cps with one or more pharmaceutical excipients;
b) optionally granulating the blend;
c) optionally blending the granules with one or more pharmaceutically acceptable inert excipients;
d) lubricating the blend of step a) or c) or granules of step b); and
e) compressing into or filling into suitable size solid dosage form.

According to yet another aspect of the present invention, there is provided a method of treatment of partial seizures with epilepsy by administering a once a day sustained release dosage form of oxcarbazepine comprising oxcarbazepine and HPMC wherein the HPMC has a viscosity of 11,000 to 25,000 cps.

According to another aspect of the present invention, there is provided a once a day sustained release dosage form of oxcarbazepine comprising oxcarbazepine and HPMC wherein the dosage form comprises:
a) about 40-70% by weight of oxcarbazepine;
b) about 20-40% by weight of hydroxypropyl methylcellulose;
c) about 1-20% by weight of diluent; and
d) about 1-20% by weight of binder;
   wherein the HPMCulose has a viscosity of 11,000 to 25,000 cps.

The term “sustained release dosage form” as used herein includes any pharmaceutical composition that achieves the slow release of drug over an extended period of time, and includes both prolonged and controlled release compositions.

The term “dosage form” as used herein includes solid dosage forms such as tablet, capsule, pill and like. The tablets can be prepared by techniques known in the art and contain a therapeutically useful amount of oxcarbazepine and such excipients as are necessary to form the tablet by such techniques. Tablets and pills can additionally be coated with functional and non-functional coatings for the purpose of acid protection, easing ability to swallow, etc.

HPMC is particularly preferred for use with oxcarbazepine because of the very low water solubility of oxcarbazepine. HPMC is available in several viscosity grades. The viscosity of the polymer controls the release rate of the drug from the formulation and affects its zero-order release kinetics.

In general, the ratio of polymer to drug is varied depending on the solubility of the drug, on the size and shape of the tablet, on the drug amount and drug release rate, and on the molecular weight and viscosity grade of the polymer.

HPMC polymers may be used as matrix components modifying the release of the drug, either alone or in combination with other materials. Oral dosage forms of the invention containing HPMC polymers may prolong drug release by forming a gelatinous matrix upon exposure to the aqueous gastrointestinal fluids which prevents or delays ingress of the aqueous gastrointestinal fluids into the dosage form and thereby prevents its rapid release. The gel matrix may be formed as a result of hydration of the HPMC polymers. In particular, HPMC having a viscosity range in between 11,000 to 25,000 cps, most preferably 11,250 to 21,000 cps, particularly approximately 15,000 cps may be used. The viscosity is determined in a 2% aqueous solution at 20° C. The concentration of HPMC may be used in the range of 15 to 50% by the total weight of composition. In particular the weight ratio of total HPMC to oxcarbazepine may range from about 1:1 to 1:20.

Preferably the oxcarbazepine is in a finely ground form, having a median particle size of approximately 2-30 μm.

The term “pharmaceutical acceptable inert excipients” as used herein includes all excipients used in the art of manufacturing solid dosage forms. Examples include binders, diluents, surfactants, lubricants/glidants, coloring agents, and the like.

Specific examples of binders include methyl cellulose, hydroxypropyl cellulose, hydroxypropyl methylcellulose, polyvinylpyrrolidone, carbomer, polysorbate 80, eudragits, gelatin, gum arabic, ethyl cellulose, polyvinyl alcohol, pullulan, pregelatinized starch, agar, tragacanth, sodium alginate, propylene glycol, and the like.

Specific examples of diluents include calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like.

Surfactants include both non-ionic and ionic (cationic, anionic and zwitterionic) surfactants suitable for use in pharmaceutical dosage forms. These include polyethoxylated fatty acids and their derivatives, for example, polyethylene glycol 400 distearate, polyethylene glycol—20 dioleate, polyethylene glycol 4-150 mono dilaurate, polyethylene glycol—20 glyceryl stearate; alcohol—oil transesterification products, for example, polyethylene glycol—6 corn oil; polyglycerized fatty acids, for example, polyglyceryl—6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol—20 sorbitan monooleate, sorbitan monolaurate; polyethylene glycol alkyl ether or phenols, for example, polyethylene glycol—20 cetyl ether, polyethylene glycol—10-100 nonyl phenol; sugar esters, for example, sucrose monopalmitate; polyoxyethylene—polyoxypropylene block copolymers known as “poloxamer”; ionic surfactants, for example, sodium caproate, sodium glycocholate, soy lecithin, sodium stearyl fumarate, propylene glycol alginate, octyl sulfosuccinate disodium, palmitoyl camitine; and the like.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like.

Besides the above mentioned excipients, other ancillary substances, in particular, coloring agents, flavoring agents, sweeteners and other additives may be used. Coloring agents of the present invention may be selected from FDA approved colorants and may be selected from Iron oxide, Lake of Tartrazine, Allura red, Lake of Quinoline yellow, Lake of Erythrosine. The sweeteners may be selected from sucrose, lactose, glucose, aspartame, saccharide or any other sweetener known from the prior art. If desired, other additives such as glidants and stabilizers can also be used.

Oxcarbazepine displays a tendency towards discoloration upon storage, hence application of a film coating may be beneficial in masking discoloration. It also may be useful to include coloring agents, e.g., pigments, in the oral dosage forms of the invention. In the case of a tablet, coloring agents may be mixed with the oxcarbazepine and tabletting excipients in the core or they may alternatively be placed solely in a coating composition, or both in the core and in the coating composition.

In a preferred embodiment according to the invention, the oral dosage form may be in the form of film-coated tablets. Conveniently, the film may be soluble in stomach juices.

For the purpose of obtaining coated tablets, the above prepared core may be coated with functional and/or non-functional layers comprising film-forming polymers, if desired. The polymer coating ingredients comprise at least one coating polymer and may include plasticizers, colorants, opacifiers, glidants, flavoring agents, diluents, fillers, bulking agents and other ingredients suitable for use in polymeric coatings.

Examples of film-forming polymers include ethylcellulose, HPMC, hydroxypropylcellulose, methylcellulose, carboxy methylcellulose, hydroxymethylcellulose, hydroxymethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit® RL and RS; and the like. Alternatively, commercially available coating compositions comprising film-forming polymers marketed under various trade names, such as Opadry® may also be used for coating. Different methods like spray coating in conventional pan, fluidized bed processor, or dip coating can be used for the coating process.

Tablets may be prepared by conventional methods like direct compression, wet granulation or dry granulation.

A polymer solution or dispersion for coating or granulation may be prepared in various solvents such as water, ethanol, methanol, isopropyl alcohol, chloroform, acetone, ether or mixtures thereof.

The following examples are representative of the invention, but are not to be construed as limiting the scope of the claims.

### EXAMPLE 1

| | | Quantity (mg/tablet) in strength | |
|---|---|---|---|
| S. No. | Ingredients | 150 mg | 300 mg |

Core tablets

| | | | |
|---|---|---|---|
| 1 | Oxcarbazepine* | 150 | 300 |
| 2 | Lactose monohydrate | 8.50 | 17.00 |
| 3 | HPMC (Methocel K15 M) | 77.50 | 155.00 |
| 4 | Colloidal Anhydrous, Silica | 2.25 | 4.50 |
| 5 | Povidone | 10.00 | 20.00 |
| 6 | Magnesium stearate | 1.75 | 3.50 |
| 7 | Isopropyl alcohol | q.s. | q.s. |
| 8 | Water | q.s | q.s |

Coating

| | | | |
|---|---|---|---|
| 9 | Opadry | 7.5 | 15.0 |
| 10 | Water | q.s. | q.s. |

| | | |
|---|---|---|
| Total tablet weight | 257.5 | 515.0 |

*Median particle size of oxcarbazepine is less than 30 μm.
1. Oxcarbazepine, lactose monohydrate, HPMC and colloidal anhydrous silica were mixed together.
2. Povidone was dissolved in a mixture of isopropyl alcohol and water to form a binder solution.
3. The blend of step 1 was granulated with the binder solution of step 2.
4. The granulate of step 3 was dried in a fluidized bed dryer.
5. The dried granules were passed through a sieve.
6. Magnesium stearate and colloidal anhydrous silica were sifted and blended with the blend of step 5.
7. The blend of step 6 was compressed into tablets.
8. A coating dispersion was prepared by dispersing Opadry in water.
9. The core tablets of step 7 were coated using the coating dispersion of step 8.

The drug release of tablets so prepared was determined by using USP apparatus 2, in a 900 ml medium containing pH 6.8 trisodium phosphate buffer with 2% sodium lauryl sulphate. The results are given in Table 1 below:

TABLE 1
Cumulative percent release of oxcarbazepine
from the tablet of Example 1.

Percent Drug Released (of label claim) (%)

| Time (h) | 150 mg | 300 mg |
|---|---|---|
| 0 | 0 | 0 |
| 1 | 9 | 7 |
| 2 | 24 | 16 |
| 4 | 40 | 34 |
| 6 | 57 | 51 |
| 8 | 73 | 67 |
| 10 | 86 | 80 |
| 12 | 96 | 95 |
| 14 | 98 | 98 |

### EXAMPLE 2

| | | Quantity (mg/tablet) in strength | |
|---|---|---|---|
| S. No. | Ingredients | 600 mg | 900 mg |

Core tablets

| | | | |
|---|---|---|---|
| 1 | Oxcarbazepine* | 600 | 900 |
| 2 | Lactose monohydrate | 35 | 52.5 |
| 3 | HPMC (Methocel K15 M) | 160 | 240 |
| 4 | Silica, Colloidal Anhydrous | 24 | 36 |
| 5 | Povidone | 5 | 7.5 |
| 6 | Magnesium stearate | 7 | 10.5 |
| 7 | Isopropyl alcohol | q.s. | q.s. |
| 8 | Water | q.s | q.s |

Coating

| | | | |
|---|---|---|---|
| 9 | Opadry | 25 | 37.5 |
| 10 | Water | q.s. | q.s. |

| | | |
|---|---|---|
| Total tablet weight | 856.00 | 1284.00 |

*Median particle size of oxcarbazepine is less than 30 μm.
1. Oxcarbazepine, lactose monohydrate, HPMC and colloidal anhydrous silica were mixed together.
2. Povidone was dissolved in a mixture of isopropyl alcohol and water to form a binder solution.
3. The blend of step 1 was granulated with binder solution of step 2.
4. The granulate of step 3 was dried in a fluidized bed dryer.
5. Dried granules were passed through a sieve.
6. Magnesium stearate was blended with the blend of step 5.
7. The blend of step 6 was compressed into tablets.
8. A coating dispersion was prepared by dispersing Opadry in water.
9. The core tablets of step 7 were coated using the coating dispersion of step 8.

The drug release of tablets so prepared was determined by using USP apparatus 2, in an 1800 ml medium containing pH 6.8 trisodium phosphate buffer with 2% sodium lauryl sulphate. The results are given in Table 2 below:

TABLE 2
Cumulative percent release from oxcarbazepine
tablet of Example 2.

Percent Drug Released (of label claim) (%)

| Time (h) | 600 mg | 900 mg |
|---|---|---|
| 0 | 0 | 0 |
| 1 | 7 | 6 |
| 2 | 16 | 14 |
| 4 | 34 | 31 |
| 6 | 51 | 47 |
| 8 | 67 | 61 |
| 10 | 79 | 75 |
| 12 | 96 | 87 |
| 14 | 98 | 97 |

### EXAMPLE 3

| | | Quantity (mg/tablet) in strength | |
|---|---|---|---|
| S. No. | Ingredients | 150 mg | 300 mg |

Core tablets

| | | | |
|---|---|---|---|
| 1 | Oxcarbazepine | 150 | 300 |
| 2 | Lactose monohydrate | 8.50 | 17.00 |
| 3 | HPMC (Methocel K15 M) | 77.50 | 155.00 |
| 4 | Povidone | 10.00 | 20.00 |
| 5 | Silica, Colloidal Anhydrous | 1.25 | 2.50 |
| | (intragranular) | | |
| 6 | Silica, Colloidal Anhydrous | 1.00 | 2.00 |
| | (extragranular) | | |
| 7 | Magnesium stearate | 1.75 | 3.50 |

Coating

| | | | |
|---|---|---|---|
| 8 | Opadry | 7.5 | 15.0 |

| | | |
|---|---|---|
| Total tablet weight | 257.5 | 515.0 |

1. Oxcarbazepine, lactose monohydrate, HPMC and intragranular colloidal anhydrous silica were mixed together.
2. Povidone was dissolved in a mixture of isopropyl alcohol and water to form a binder solution.
3. The blend of step 1 was granulated with binder solution of step 2.
4. The granulate of step 3 was dried in a fluidized bed dryer.
5. Dried granules were passed through sieve.
6. Magnesium stearate and extragranular colloidal silica were blended with the blend of step 5.
7. The blend of step 6 was compressed into tablets.
8. A coating dispersion was prepared by dispersing Opadry in water.
9. The core tablets of step 7 were coated using the coating dispersion of step 8.

### EXAMPLE 4

This composition is prepared per the composition given for Example 1, wherein the median particle size is 19-25 μm.

### EXAMPLE 5

This composition is prepared per the composition given for Example 2, wherein the median particle size is 19-25 μm.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim

1. A once a day sustained release dosage form comprising oxcarbazepine and hydroxypropyl methylcellulose (HPMC), wherein the HPMC has a viscosity of 11,000 to 25,000 cps.

2. The sustained release dosage form according to claim 1, wherein the concentration of hydroxypropyl methylcellulose comprises 10 to 50% of the total composition.

3. The sustained release dosage form according to claim 1, wherein the median particle size of oxcarbazepine is less than 30 μm.

4. The sustained release dosage form according to claim 1, wherein the dosage form comprises one or more pharmaceutically acceptable inert excipients selected from diluents, binders, disintegrants, surfactants, lubricants/glidants, and coloring agents.

5. The sustained release dosage form according to claim 1, wherein the dosage form comprises a tablet, capsule or pill.

6. The sustained release dosage form of claim 5, wherein the dosage form is further coated with a functional or nonfunctional coating.

7. The sustained release dosage form of claim 1, comprising
a) about 40-70% by weight of oxcarbazepine,
b) about 20-40% by weight of HPMC,
c) about 1-20% by weight of diluent,
d) about 1-20% by weight of binder.
wherein the HPMC has a viscosity of 11,000 to 25,000 cps.

8. A process for the preparation of once a day sustained release dosage form of oxcarbazepine, the process comprising the steps of
a) blending oxcarbazepine and HPMC with one or more pharmaceutically acceptable inert excipients,
b) optionally granulating the blend,
c) optionally blending the granules with one or more pharmaceutically acceptable inert excipients,
d) lubricating the blend of step a) or c) or granules of step b), and
e) compressing into or filling into suitable size solid dosage form, wherein the HPMC has a viscosity of 11,000 to 25,000 cps.

9. The process according to claim 8, wherein the granulation comprises wet granulation or dry granulation.

10. The process according to claim 9, wherein the wet granulation comprises water or suitable organic solvent or a mixture thereof.

11. A method of treating partial seizures with epilepsy comprising administering to the mammal an effective amount of a sustained release dosage form of oxcarbazepine according to claim 1.
